(19) United States
(12) Patent Application Publication
(10) Pub. No.: US 2010/0291614 A1
(43) Pub. Date: Nov. 18, 2010

(54) IN VITRO METHOD FOR DETERMINING THE RISK OF AND DIAGNOSIS OF AN ARTERIAL VASCULAR DISORDER

(76) Inventors: Wolfgang Nitz, Buch (DE); Andreas Rechner, Marburg (DE)

Correspondence Address: HARNESS, DICKEY & PIERCE, P.L.C., P.O.BOX 8910, RESTON, VA, 20195, US

(21) Appl. No.: 12/780,155

(22) Filed: May 14, 2010

(30) Foreign Application Priority Data
May 18, 2009 (DE) 10 2009 021 742.8

(51) Int. Cl. C12Q 1/02

(52) U.S. Cl. 435/29

(57) ABSTRACT

An in vitro method is disclosed for (i) determining the risk to an individual of developing an arterial vascular disorder, or (ii) diagnosing an arterial vascular disorder. In at least one embodiment, the method includes at least the following: (a) determining at least one biomarker characteristic of a vascular disorder and/or an early stage of an inflammation in a biological sample of an individual, (b) determining the state of a vessel displayed using an imaging method, wherein the information obtained in (a) and (b) is correlated with (i) or (ii).

### PRIORITY STATEMENT

The present application hereby claims priority under 35 U.S.C. §119 on German patent application number de 10 2009 021 742.8 filed May 18, 2009, the entire contents of which are hereby incorporated herein by reference.

### FIELD

At least one embodiment of the invention generally relates to an in vitro method and/or a system for determining the risk to an individual of developing an arterial vascular disorder and the evaluation and analysis of this risk. At least one embodiment of the invention furthermore generally relates to an in vitro method for diagnosing a vascular disorder.

### BACKGROUND

Vascular disorders are amongst the most widespread disorders world-wide and will increase significantly in the future. The demographic development in the industrialized nations and the increase in diabetes disorders in particular are the main reasons for this development. Vascular disorders are any type of pathological change to the vessels of an organism. These changes can affect practically every part of the organism. The main consequences of vascular disorders are strokes, myocardial infarcts, circulatory disorders in the legs, and, in the worst case scenario, leg amputations or even death. Vascular disorders and the effects thereof on the health of a human are difficult to cure and connected to high costs for the health system. The main risk factors of vascular disorders include high blood pressure, lipopathies, smoking and, in particular, diabetes mellitus. Prevention, early detection, a targeted and in particular timely diagnosis and an adapted therapy, and, if need be, rehabilitation play an outstanding role in combating vascular disorders and the consequences thereof.

Currently, the risk of a patient suffering from a vascular event (for example myocardial infarct, stroke) is evaluated using so-called risk scores (for example Framingham, PROCAM, ESC, American Heart Association). These consider the following risk factors: age, smoking, high blood pressure, lipopathy, occurrence of a vascular disorder in the family, post-menopause in the case of women, diabetes mellitus, major obesity, and lack of exercise. In general, a patient has to exhibit at least two or three of the nine risk factors to be considered a patient with an elevated risk. However, the presence of two or more risk factors for a vascular disorder does not automatically result in a preventative treatment, which could in some cases be advantageous to the patient.

The diagnosis of a vascular disorder is currently based on the medical history and the commonly unspecific symptoms of the patient. If there are doubts relating to the diagnosis or if there are complications during the treatment, there is a referral to a specialist or consultant for further clarification. The further treatment is then based on the findings of the consultant. The disadvantage of this procedure to the patient is that some additional risk factors are recognized either too late or not at all, that early stages of a vascular disorder are not recognized and treated in time, and that valuable time often passes before an adequate treatment of an already present vascular disorder is initiated.

### SUMMARY

Thus, the inventors of the present application discovered that there is a need for an effective and reliable method for diagnosing a vascular disorder. The method should potentially allow more targeted and effective therapy or prevention by recording various aspects of the disorder. Thus, the method should also allow determination of the risk to an individual of developing a vascular disorder. Furthermore, the method should record, as a whole, the presence or the possibility of the presence of a vascular disorder. The method should allow statements regarding targeted prevention or therapy of a vascular disorder. Furthermore, it should be possible to carry out the method in a simple and cost-effective fashion. It should also be suitable for routine examinations and not be stressful for the patient. Furthermore, it should be possible to carry out the method on an outpatient basis.

Now, it was surprisingly discovered that an effective and efficient diagnosis of a vascular disorder is allowed by a combination of determining biomarkers connected to a vascular disorder and/or an early stage of the inflammation in a biological sample and an imaging method for displaying the endothelial dysfunction or for evaluating the vessel displayed in an imaging method in respect of endothelial dysfunctions.

Endothelial dysfunctions, as can be documented using imaging methods, are often interpreted as the after effect of biochemical changes. In contrast thereto, at least one embodiment of the present invention allows the prognosis that in the case of many cardiovascular disorders, in which a risk to the patient was identified by in vitro diagnostics, it is only the endothelial dysfunction documented by the imaging method that allows estimating the risk to the patient.

At least one embodiment of the invention is directed to an in vitro method for (i) determining the risk to an individual of developing an arterial vascular disorder or (ii) diagnosing an arterial vascular disorder, the method comprises at least the following steps: (a) determining at least one biomarker characteristic of a vascular disorder and/or an early stage of an inflammation in a biological sample of an individual, (b) determining the state of a vessel displayed using an imaging method, wherein the information obtained in (a) and (b) is correlated with (i) or (ii).

In the case of the method according to at least one embodiment of the invention, at least one biomarker characteristic of a vascular disorder and/or an early stage of an inflammation is or are firstly determined in a biological sample of an individual. The biological sample can be any biological material that can be taken from an organism and in which the aforementioned biomarkers can be detected. The biological sample is preferably a blood sample. The biomarkers characteristic of a vascular disorder are preferably biomarkers indicative of hemostasis and biomarkers indicative of lifestyle. Biomarkers indicative of hemostasis are for example the closure time of the PFA-100 system, in which the time is determined that the thrombocytes (blood platelets) of a whole blood sample require to close off an aperture with a defined diameter. Such tests provide information in respect of a dysfunction of the primary hemostasis, which leads to hypofunction or hyperfunction of the thrombocytes. Further examples of a biomarker to be determined according to the invention are clotting factors, which provide information in respect of the individual clotting status.

A further example is determining the clotting potential, for example by determining the endogenous thrombin potential (ETP). By way of example, the clotting status can be determined using the clotting test ProC® Global Assay, which determines global dysfunctions in the protein C system of the clot. However, it is also possible to use other commercially available clotting tests.

A further example for globally recording the hemostasis is thromboelastography, in which, for example, the clotting profile of a recalcified whole blood sample is recorded and analyzed.

Such tests are commercially available and known to a person skilled in the art. These tests are conducted according to the specifications of the manufacturer or generally known recommendations in the art.

Biomarkers indicative of lifestyle are, for example, biomarkers which provide information about dietary habits such as the plasma levels of L-ascorbic acid and/or beta carotene and/or alpha-tocopherol and/or folic acid and/or vitamin B12 and/or selenium, and the cholesterol level, the triglyceride level, or the levels of LDL cholesterol and HDL cholesterol. Biomarkers indicative of lifestyle are, for example, biomarkers for oxidative stress, such as the plasma level of oxidized LDL, the concentration of the lipid peroxidation products malondialdehyde (MDA) and/or 4-hydroxyalkenal (HAE) and/or hexanoyl-lysine (HEL) and/or 4-hydroxynonenal (4-HNE) and/or acrolein, the concentration of homocysteine or myeloperoxidase (MPO) in serum or plasma, the concentration of glutathione peroxidase in cell extracts, the concentration of 8-hydroxy-2′-deoxyguanosine (8-OHdG), a marker for the oxidative damage to DNA, and the concentration of aldehyde-modified serum proteins (protein carbonyls), which are a marker for the oxidation of protein. Global biomarkers indicative of lifestyle are, for example the redox potential or the redox state or the antioxidative capacity of blood or plasma. Furthermore, these are biomarkers that are indicative of the functionality of the endothelium, such as the plasma or serum levels of asymmetric dimethylarginine (ADMA).

Determining the aforementioned parameters is well-known in the art. Appropriate tests are commercially available. The tests are conducted according to the specifications of the manufacturer or generally recognized recommendations in the art.

Examples of markers of an early stage of an inflammation are the C-reactive protein (CRP), interleukin 6 (IL-6) or procalcitonin (PCT), furthermore heteropolymer of the myeloid-related protein MRP8/14 (MRP8/14), monocyte chemotactic protein MCP3 (MCP-3), serum amyloid A (SAA), interleukin-1beta (IL-1β), tissue necrosis factor alpha (TNF-alpha), vascular endothelial growth factor C (VEGF-C), nitrotyrosine, E-selectin, L-selectin, collagenases or pentraxin 3 (PTX3/TSG-14).

Tests for determining the aforementioned markers are well-known in the art and are commercially available. The tests are conducted according to the specifications of the manufacturer or generally recognized recommendations in the art.

Furthermore, according to at least one embodiment of the invention, the state of a vessel displayed using an imaging method is evaluated. Here, this is preferably the display of the arterial vascular elasticity. A method for documenting the loss of elasticity of an artery wall is the detection of the thickening of the intima media in an arterial vessel. Here, high-resolution (intravascular) sonography was suggested as imaging method, but the vessel wall can also be displayed at a similar resolution with the aid of a magnetic resonance imaging method. Many studies indicate a relationship between arterial vessel wall thickness and the frequency of a cardiovascular event. It is known that high blood pressure can constitute a measurable symptom of a vascular disorder. Thus, e.g. modern antihypertensive agents are required not only to lower the blood pressure, but also to develop a vessel-protective effect. Measuring the structural changes on the vessel walls already had made it possible to document that e.g. sartans lead to a detectable regression of pathological vessel wall changes, whereas this could not be detected for e.g. diuretics and beta blockers.

A further method of at least one embodiment is for determining the arterial vascular elasticity based on an imaging method is the measurement of the aortic elasticity. This method preferably uses gradient echo or SSFP MRI sequences with a high time-resolution generally triggered with the heart beat, showing recordings perpendicular to an arterial vessel and allowing conclusions to be drawn in respect of the aortic elasticity by evaluating the change in the diameter of the arterial vessel over the cardiac cycle. Previously published work from academia shows a strong age-dependence of the aortic elasticity and a correlation with the degree of arteriosclerosis present.

A further method of at least one embodiment is for determining the arterial vascular elasticity based on an imaging method is the measurement of the pulse-wave speed using MRI. Academic studies publicize a correlation between the pulse-wave speed and the aortic elasticity. As the elasticity of the arterial vessel decreases, the pulse-wave speed increases. Temporal high-resolution phase-contrast MRI triggered by the heart beat at various slice planes perpendicular to the arterial vessel allows the calculation of the pulse-wave speed by means of the time difference between the pulse-wave arrival at the various slices and the known slice spacing, and the compliance can be calculated therefrom as a parameter of the aortic elasticity.

A further method of at least one embodiment is for determining the arterial vascular elasticity based on an imaging method is the measurement of the pulse-wave speed using MRI, wherein the blood-flow speed and hence the pulse-wave arrival is determined by so-called Fourier encoding.

A further method of at least one embodiment is for determining the arterial vascular elasticity based on an imaging method is the measurement of the pulse-wave speed using MRI, wherein the blood-flow speed and hence the pulse-wave arrival are determined by the temporal change of a marked blood bolus (tagging, labeling).

A further method of at least one embodiment is for determining the arterial vascular elasticity based on an imaging method is the measurement of the pulse-wave speed using MRI, wherein the blood-flow speed and hence the pulse-wave arrival is determined by the temporal change of a forming equilibrium state in the case of an SSFP sequence (GCFP-global coherent free precession).

A further method of at least one embodiment is for determining the arterial vascular elasticity, including the precapillary vessel resistance, based on an imaging method is the analysis of the pulse-wave shape with respect to the pulse-wave reflection. Here, the pulse-wave shape can be determined with the aid of one of the methods cited above or in the following text.

A further method of at least one embodiment is for determining the arterial vascular elasticity based on an imaging method is the measurement of the pulse-wave speed using Doppler sonography.

A further method of at least one embodiment is for directly determining the arterial vascular elasticity based on an imaging method is the direct measurement of the vascular elasticity with the aid of MR elastography.

A further method of at least one embodiment is for directly determining the arterial vascular elasticity based on an imaging method is the direct measurement of the vascular elasticity with the aid of vibro-acoustography.

A further method of at least one embodiment is for determining the arterial vascular elasticity based on an imaging method is the measurement of the pulse-wave speed under conventional X-ray irradiation by following the distribution of a contrast-agent bolus over time.

Equipment for some of the aforementioned applications is commercially available and its application is generally known.

Other described methods can be implemented on existing equipment and are in the scientific-trial phase. They are thus available to a person skilled in the art.

It was found that the elasticity of the arteries and the stiffness of the aorta in particular is a further risk factor for a vascular disorder. However, the significance of this risk factor is synergistically increased by evaluating the determination of the aforementioned biomarkers from a biological sample. Conversely, the significance of the biomarkers to be determined in a biological sample of an individual is synergistically increased by evaluating the data relating to the vessel state obtained by an imaging method. The combined evaluation of the determination of the aforementioned biomarkers from a biological sample and the evaluation of the data relating to the vessel state obtained by an imaging method, in particular the arterial elasticity, thus allow a risk evaluation or a diagnosis of a vascular disorder, the significance and the accuracy of which synergistically exceeds the significance of the respective individual examinations.

Particularly advantageous results can be obtained with the following combinations of biomarkers and imaging methods:

1 biomarker relating to dietary habits+1 biomarker from oxidative stress+1 biomarker from inflammatory processes+1 imaging method;

Example: plasma level of L-ascorbic acid, homocysteine and CRP combined with high-resolution sonography measurements of the wall thickness of the aorta or carotid artery,

or the redox state of the blood+the state of the primary hemostasis+1 biomarker from inflammatory processes+1 imaging method;

Example: antioxidative capacity of plasma/serum+results from the Col/ADP measurement cell of a whole blood sample in the PFA-100 system+plasma level of CRP or Pentraxin 3+ determining the flexibility or wall thickness of an arterial vessel wall by means of a suitable imaging method.

In the case of the reduction in the arterial vessel elasticity documented by the imaging system, an elevated LDL/HDL cholesterol value constitutes an elevated risk compared to arterial vessel elasticity without pathological findings.

In the case of the reduction in the arterial vessel elasticity documented by the imaging system, an elevated CRP value as marker for inflammatory processes constitutes an elevated risk compared to arterial vessel elasticity without pathological findings.

The method according to at least one embodiment of the invention allows the recording of vascular disorders in their entirety. According to at least one embodiment of the invention, the presence of a vascular disorder can also be determined in good time before the occurrence of clinically relevant symptoms and thus effective prevention or therapy can be initiated. The method according to at least one embodiment of the invention also allows reliable determination of patients at risk, who can thus be subjected to regular and targeted follow-up examinations. This can achieve effective prevention in the field of vascular disorders and thus vascular disorders and their subsequent disorders, or even deaths as a result thereof, can be minimized or avoided.

The diagnostic or prognostic value of the method according to at least one embodiment of the invention can be increased by including the evaluation of the aforementioned conventional risk factors for vascular disorders.

Determining at least one biomarker characteristic of a vascular disorder and/or an early stage of an inflammation and determining the status of a vessel displayed by way of an imaging method results in the following risk assessment:

The results of the combined methods are conveniently evaluated by assigning a score such as points or numbers or symbols to each value of the individual methods lying in a specified range. Here, the specific range is characterized by displaying a pathological or abnormal change in the measurement variable. It is also possible for a plurality of regions to be defined for the results of a method, which regions differ in the severity of the pathological change and are then assigned correspondingly different scores. The scores of the individual methods of a combination are then summed to form an overall value. A higher overall value of a combination of the methods means that the vascular disorder is rated more severe and the risk of a vascular event is rated higher. The level of the overall value could then allow the derivation of expedient therapeutic measures, such as the recommendation of a change in lifestyle or the preventative dispensation of e.g. vitamin preparations, acetylsalicylic acid, antihypertensive agents or statin derivatives.

According to at least one embodiment of the invention, the evaluation is brought about either manually by a trained medical practitioner or by a computer program.

The method according to at least one embodiment of the invention can also be carried out as a method in which the individual to be examined is firstly subjected to an imaging method and the images obtained thereby are then evaluated by a medical practitioner.

The method according to at least one embodiment of the invention can diagnose arterial vascular disorders in a targeted fashion, or a risk thereof can be determined. Examples of such arterial vascular disorders include arteriosclerosis, arterial occlusive disease, coronary vascular disease, transient ischemic attacks (TiA), stroke, myocardial infarction, angina pectoris, circulatory disorders, in particular peripheral circulatory disorders.

At least one embodiment of the invention relates to a system for
(i) determining the risk to an individual of developing an arterial vascular disorder, or
(ii) diagnosing an arterial vascular disorder, within the scope of an examination of a patient to be conducted, comprising a data processing device with stored program segments designed for executing a method as claimed in one of the preceding claims.

In such a system, the entirety of the information can continuously be expanded by newly obtained information as a result of performed examinations. This information is taken into account by the system, which now compensates for and processes all the information available thereto.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

An embodiment of the present invention is directed to an in vitro method for (i) determining the risk to an individual of developing an arterial vascular disorder, or
(ii) diagnosing an arterial vascular disorder. In at least one embodiment, the method comprises at least the following steps:
(a) determining at least one biomarker characteristic of a vascular disorder and/or an early stage of an inflammation in a biological sample of an individual,
(b) determining the state of a vessel displayed using an imaging method,
   wherein the information obtained in (a) and (b) is correlated with (i) or (ii).

An embodiment of the invention relates to a system for
(i) determining the risk to an individual of developing an arterial vascular disorder, or
(ii) diagnosing an arterial vascular disorder, within the scope of an examination of a patient to be conducted, comprising a data processing device with stored program segments designed for executing a method as described in at least one of the embodiments of the present application.

In such a system, the entirety of the information can continuously be expanded by newly obtained information as a result of performed examinations. This information is taken into account by the system, which now compensates for and processes all the information available thereto.

The system can automatically correlate the data from (a) determining at least one biomarker characteristic of a vascular disorder and/or an early stage of an inflammation in a biological sample of an individual and (b) determining the state of a vessel displayed using an imaging method for the purpose of determining a risk and/or a diagnosis. By way of example, current results can be compared to information from a database.

Within the scope of processing the data, it is furthermore expedient for information relevant to the examination and/or diagnosis to be selected and output in addition to a utilized examination modality (imaging and/or in vitro). This information can be, for example, guidelines from for example the medical council, earlier case studies or current citations etc., that is to say any additional information that can assist the medical practitioner within the scope of the upcoming examination.

It is preferable, in at least one embodiment, for there to be a communication link between the data processing device and an image archiving and patient information system.

Furthermore, there can be a communication link between the data processing device and a further data processing and/or display device, on which the diagnosis is performed or the risk is determined, e.g. a decentralized computer terminal with a display device connected to the central data processing device.

An embodiment of the invention will be explained in more detail on the basis of the following example.

### EXAMPLE

The patient has at least two of the eight risk factors for vascular disorders but does not yet exhibit symptoms or discomfort. Routine blood pressure measurement is combined with a flow-dilation measurement. A blood sample is taken on an empty stomach and it is immediately analyzed by trained personnel as follows:
1. PFA-100 measurement using the Col/ADP cartridge (whole blood)
2. Photometric determination of the plasma level of L-ascorbic acid
3. Photometric determination of the antioxidative capacity of the plasma or the plasma level of thiobarbituric acid reactive substances
4. Photometric determination of the CRP level

All photometric determinations can be carried out by small, specific, fully-automated analysis equipment.

The results are automatically analyzed by a computer program, which assigns a factor for the vascular health. By way of example, in the process, closing times of the PFA-100 Col/ADP measurement cell <70 s, plasma levels of L-ascorbic acid <6 mg/L, TBARS >4 μM and CRP >3 mg/L and the thickness of the intima media >0.8 mm, measured by high-resolution sonography, are each assigned a positive score, e.g. 1 point. Furthermore, closing times of the PFA-100 Col/ADP measurement cell <64 s, plasma levels of L-ascorbic acid <4 mg/L, TBARS >6 μM and CRP >10 mg/L and the thickness of the intima media >1.2 mm, measured by high-resolution sonography, are each assigned a higher positive score, e.g. 2 points. The factor for the vascular health is then calculated from the positive scores of all methods of the combinations. By way of example, if a patient then reaches a factor of four or more points with this combination of methods, this indicates that preventative measures such as a change in lifestyle or the dispensation of vitamin preparations, acetylsalicylic acid, antihypertensive agents or statin derivatives would be expedient. Which measures are particularly indicated can be also be derived from the results of the individual measurement methods, for example the targeted dispensation of antioxidants such as L-ascorbic acid or vitamin E in the case of very high TEARS values.

The effectiveness of the measures can be monitored using the same method. Thus, conducting the method according to an embodiment of the invention later can monitor whether, for example, the factor of the vascular health is reduced to below the critical value of four points.

The aforementioned boundaries, the automatic evaluation of the measurement values and the calculation and evaluation of the factor of the vascular health are expediently stored in a computer program for calculating the overall factor.

The object is the reduction in the incidence of a vascular disorder before the occurrence of severe symptoms or vascular events by way of timely and targeted therapy.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, computer readable medium and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable medium and is adapted to perform any one of the aforementioned methods when run on a computer device (a device including a processor). Thus, the storage medium or computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An in vitro method for
(i) determining a risk to an individual of developing an arterial vascular disorder, or
(ii) diagnosing an arterial vascular disorder, the method comprising:
(a) determining at least one biomarker characteristic of at least one of a vascular disorder and an early stage of an inflammation in a biological sample of an individual; and
(b) determining the state of a vessel displayed using an imaging method, wherein information obtained from the determinations in (a) and (b) is correlated with (i) or (ii).

2. The method as claimed in claim 1, further comprising evaluating the following risk factors of an individual: age, smoking, high blood pressure, hyperlipidemia, occurrences of vascular disorders in the family, post-menopause in the case of a woman, diabetes mellitus, obesity, lack of exercise.

3. The method as claimed in claim 1, wherein the at least one biomarker characteristic of a vascular disorder is selected from biomarkers indicative of hemostasis and biomarkers indicative of lifestyle.

4. The method as claimed in claim 1, wherein the at least one biomarker characteristic of an early stage of an inflammation is selected from C-reactive protein (CRP), interleukin 6 (IL-6) or procalcitonin (CPT), a heteropolymer of the myeloid-related protein MRP8/14, monocyte chemotactic protein MCP-3, serum amyloid A (SAA), interleukin-1beta (IL-1β), tissue necrosis factor alpha (TNF-alpha), vascular endothelial growth factor C (VEGF-C), nitrotyrosine, E-selectin, L-selectin, collagenases or pentraxin 3 (PTX3/TSG-14).

5. The method as claimed in claim 1, wherein the method used to image the vessel is selected from magnetic resonance phase velocity encoding, magnetic resonance Fourier velocity encoding, magnetic resonance tagging, magnetic resonance 1D single or multislice displacement method, magnetic resonance global coherent free precession imaging, ultrasonography, imaging angiography with a contrast agent, Doppler ultrasound displacement measurement, ultrasound vibro-acoustography and MR elastography.

6. The method as claimed in claim 3, wherein the at least one biomarker indicative of the hemostasis is obtained from global hemostasis testing, global thrombocyte function testing, testing the clotting potential, or global testing of the protein C system.

7. The method as claimed in claim 3, wherein the at least one biomarker indicative of lifestyle is selected from L-ascorbic acid, beta carotene, alpha, tocopherol, folic acid, vitamin B12, selenium, cholesterol, triglycerides, LDL cholesterol and HDL cholesterol, malondialdehyde (MDA), 4-hydroxyalkenal (HAE), hexanoyl-lysine (MEL), 4-hydroxynonenal (4-HNE), acrolein, homocysteine, myeloperoxidase (MPO), glutathione peroxidase, 8-hydroxy-2′-deoxyguanosine (8-OHdG), aldehyde-modified serum proteins (protein carbonyls), asymmetric dimethylarginine (ADMA) or the redox potential or redox state or the antioxidative capacity of the blood.

8. The method as claimed in claim 1, wherein the vascular disorder is selected from arteriosclerosis, coronary vascular disease, peripheral artery occlusive disease, transient ischemic attack (TiA), stroke, myocardial infarction, angina pectoris and circulatory disorder.

9. A system for
(i) determining the risk to an individual of developing an arterial vascular disorder, or
(ii) diagnosing an arterial vascular disorder, within the scope of an examination of a patient to be conducted, comprising:
a data processing device with stored program segments designed for executing the method as claimed in claim 1.

10. The system as claimed in claim 9, further comprising a communication link between the data processing device and an image archiving and patient information system.

11. The system as claimed in claim 9, further comprising a communication link between the data processing device and a data processing and display device, on which the diagnosis is performed or the risk is determined.

12. The method as claimed in claim 2, wherein the at least one biomarker characteristic of a vascular disorder is selected from biomarkers indicative of hemostasis and biomarkers indicative of lifestyle.

13. The method as claimed in claim 2, wherein the at least one biomarker characteristic of an early stage of an inflammation is selected from C-reactive protein (CRP), interleukin 6 (IL-6) or procalcitonin (CPT), a heteropolymer of the myeloid-related protein MRP8/14, monocyte chemotactic protein MCP-3, serum amyloid A (SAA), interleukin-1beta (IL-113), tissue necrosis factor alpha (TNF-alpha), vascular endothelial growth factor C (VEGF-C), nitrotyrosine, E-selectin, L-selectin, collagenases or pentraxin 3 (PTX3/TSG-14).

14. The method as claimed in claim 2, wherein the method used to image the vessel is selected from magnetic resonance phase velocity encoding, magnetic resonance Fourier velocity encoding, magnetic resonance tagging, magnetic resonance 1D single or multislice displacement method, magnetic resonance global coherent free precession imaging, ultrasonography, imaging angiography with a contrast agent, Doppler ultrasound displacement measurement, ultrasound vibro-acoustography and MR elastography.

15. A computer readable medium including program segments for, when executed on a computer device, causing the computer device to implement the method of claim 1.

16. The method of claim 1, wherein a data processing device is used to perform at least one of the determining and diagnosing.
